(19) United States
(12) Patent Application Publication
(10) Pub. No.: US 2024/0066292 A1
(43) Pub. Date: Feb. 29, 2024

(54) Neurological Stimulation and Signal Recording Apparatus

(71) Applicant: Terry Daglow, Houston, TX (US)

(72) Inventor: Terry Daglow, Houston, TX (US)

(21) Appl. No.: 18/457,204

(22) Filed: Aug. 28, 2023

Related U.S. Application Data
(60) Provisional application No. 63/401,682, filed on Aug. 28, 2022.

(51) Int. Cl. A61N 1/05; A61N 1/375

(52) U.S. Cl. CPC A61N 1/0551; A61N 1/375

(57) ABSTRACT

A neurostimulation lead for stimulating neural tissue of a patient. The neurostimulation lead includes a lead body comprising insulative material, a plurality of electrodes, a plurality of terminals, a plurality of conductors. The plurality of electrodes may be electrically coupled to the plurality of terminals through coupling of the plurality of conductors which are disposed along a length of the lead body.

### CROSS-REFERENCE TO RELATED APPLICATIONS

This application claims priority to U.S. Provisional Patent Application 63/401,682, filed on Aug. 28, 2022.

### STATEMENT REGARDING FEDERALLY SPONSORED RESEARCH

Not applicable.

### BACKGROUND

Neurostimulation systems generally include a pulse generator and one or more leads. The pulse generator is typically implemented using a metallic housing that encloses circuitry for generating the electrical pulses, control circuitry, communication circuitry, a rechargeable battery, etc. The pulse generation circuitry is coupled to one or more stimulation leads through electrical connections provided in a “header” or the pulse generator. Stimulation leads typically include multiple wire conductors enclosed or embedded within a lead body or insulative material. Terminals and electrodes are located on the proximal and distal ends of the leads. The conductors or the leads electrically couple the terminals to the electrodes. The electrical pulses from the pulse generator are conducted through the leads and applied to patient tissue by the electrodes or the leads.

In general, the invention is a device and apparatus incorporating a novel approach for joining the terminal and electrode to a conductor and a method for fabricating this novel approach into an assembly of multiple terminal, electrode, and conductor assemblies.

### SUMMARY

An apparatus is conceived for the manufacture of the device. It contains unique and novel elements for the joining of terminal and electrodes to a conductor and the construction of the electrical device.

Some embodiments of the neurostimulation lead tool include a lead body comprising a terminal end and an electrode end; said terminal end is comprised of a terminal band, an inner band, an electrical conductor, wherein said electrical conductor has a proximal end and a distal end, wherein said terminal band has an inner surface and outer surface and said inner band has an outer surface, wherein said proximal end of said electrical conductor is in electrical communication with said terminal band when said electrical conductor is disposed between said terminal band and said inner band; said electrode end is comprised of an electrode band, an inner band, and the distal end of said electrical conductor, wherein said electrode band has an inner surface and outer surface and said inner band has an outer surface, wherein said distal end of said electrical conductor is in electrical communication with said terminal band when said electrical conductor is disposed between said electrode band and said inner band.

Some embodiments of the neurostimulation lead tool include an electrical conductor that is coated with an electrically insulative material. Some embodiments of the neurostimulation lead tool include a polymer substrate. Some embodiments of the neurostimulation lead tool include a portion of the electrically insulative material coating of said proximal end of electrical conductor is removed from the electrical conductor by the mating of said inner surface of said terminal band with said outer surface of said inner band thereby facilitating an electrical connection between said proximal end of said electrical conductor and said terminal band. Some embodiments of the neurostimulation lead tool include a portion of the electrically insulative material coating of said distal end of said electrical conductor is removed from the electrical conductor by the mating of said inner surface of said electrode band with said outer surface of said inner band thereby facilitating an electrical connection between said distal end of said electrical conductor and said electrode band. Some embodiments of the neurostimulation lead tool include a portion of the electrically insulative material coating of said electrical conductor is removed before assembly of said electrical conductor, said terminal band, and said inner band thereby facilitating an electrical connection between said proximal end of said electrical conductor and said terminal band. Some embodiments of the neurostimulation lead tool include wherein a portion of the electrically insulative material coating of said electrical conductor is removed before assembly of said electrical conductor, said electrode band, and said inner band thereby facilitating an electrical connection between said distal end of said electrical conductor and said electrode band.

Some embodiments of the neurostimulation lead tool include an electrical conductor that is fastened to said terminal band or said electrode band by one of the following: weld, laser weld, resistance spot weld, or solder.

Some embodiments of the neurostimulation lead tool include a lead body comprising a proximal end and a distal end; a plurality of terminal bands disposed at the proximal end of said lead body; a plurality of electrode bands disposed at the distal end of said lead body; a plurality of electrical conductors electrically connecting each of said terminal bands to a respective one of said electrode bands; a polymer substrate isolating the plurality of terminal bands from each other terminal band and further isolating the plurality of electrode bands from each other electrode band and further isolating the plurality of electrical conductors from each other electrical conductors to form a plurality of electrically communicative terminal bands with respective electrode bands.

Some embodiments of the neurostimulation lead tool include a method of manufacturing a neurostimulation lead comprising the steps of: electrically connecting a terminal end of a lead body with an electrode end of said lead body, wherein said terminal end is comprised of a terminal band, an inner band, an electrical conductor, wherein said electrical conductor has a proximal end and a distal end, wherein said terminal band has an inner surface and outer surface and said inner band has an outer surface, wherein said proximal end of said electrical conductor is in electrical communication with said terminal band when said electrical conductor is disposed between said terminal band and said inner band; wherein, said electrode end is comprised of an electrode band, an inner band, and the distal end of said electrical conductor, wherein said electrode band has an inner surface and outer surface and said inner band has an outer surface, wherein said distal end of said electrical conductor is in electrical communication with said terminal band when said electrical conductor is disposed between said electrode band and said inner band; placing said lead body into a mold assembly fixture having a cavity; placing a plurality of plates on said terminal end of said mold assembly fixture; placing a plurality of plates on said electrode end of said mold assembly fixture; insertion of guide pins through mold assembly fixture and said plurality of plates on said terminal end and said electrode end of said mold assembly fixture; placement of a spacer component around said plurality of plates at said terminal end or said electrode end to provide offsets or alignment for said plurality of plates; injecting a polymer material into said cavity of said mold assembly fixture to form a polymer substructure around the electrically connected terminal end of said lead body and said electrode end of said lead body.

Some embodiments of the neurostimulation lead tool include the method of manufacturing said neurostimulation lead wherein said electrical conductor is fastened to said terminal bands or said electrode bands by one of the following: weld, laser weld, resistance spot weld, or solder. Some embodiments of the neurostimulation lead tool include the method of manufacturing said neurostimulation lead of claim 17 further comprising the step of injecting another layer of polymer material to provide an overmold layer of polymer material.

Some embodiments of the neurostimulation lead tool include a second layer of polymer material over a polymer substrate.

### BRIEF DESCRIPTION OF THE DRAWINGS

FIG. 1A depicts a completed conductor lead.

FIG. 1B depicts a close-up view of a distal end of the completed conductor lead depicted in FIG. 1A.

FIG. 1C depicts a close-up view of a proximal end of the completed conductor lead depicted in FIG. 1A.

FIGS. 2A, 2B, and 2C depict an isometric view of an embodiment and method for assembly using an insulation displacement method for electrically connecting a lead conductor to one or more terminal bands.

FIGS. 3A, 3B, and 3C depict an isometric view of an embodiment and method for assembly using a pre-stripped insulation method for electrically connecting a lead conductor to one or more terminal bands.

FIG. 4A depicts an isometric view of the lead conductor in an assembled state.

FIG. 4B depicts an exploded, isometric view of the lead conductor shown in FIG. 4A.

FIG. 4C depicts an exploded, close-up view of the lead conductor shown in FIG. 4B.

FIG. 4D depicts an exploded, close-up view of a distal end of the lead conductor shown in FIG. 4C.

FIG. 4E depicts an exploded, close-up view of a proximal end of the lead conductor shown in FIG. 4C.

FIG. 5A depicts an orthographic section view of an embodiment of an assembly terminal band, an electrode, and a conductor prior to mating of a substructure to the assembly.

FIG. 5B depicts an orthographic cross-section view of an embodiment of an assembly terminal band, an electrode, and a conductor prior to mating of a substructure to the assembly.

FIG. 5C depicts an orthographic cross-section close-up view of an embodiment of an assembly shown in FIG. 5B.

FIG. 5D depicts an orthographic cross-section close-up view of an embodiment of an assembly shown in FIG. 5B.

FIG. 6A depicts an isometric view of a lead assembly after mating with a substructure.

FIG. 6B depicts an isometric view of a distal end of a lead assembly after mating with a substructure as shown in FIG. 6A.

FIG. 6C depicts an isometric view of a proximal end of a lead assembly after mating with a substructure as shown in FIG. 6A.

FIG. 7A is an isometric, front view depicting an embodiment of a conductor assembly showing a relationship between one or more terminal bands, electrodes, and conductors.

FIG. 7B is an isometric, cross-sectional, front view depicting the embodiment shown in FIG. 7A.

FIG. 7C is an isometric, front view of an access port for joining of a trimmed conductor of the embodiment depicted in FIGS. 7A and 7B.

FIG. 7D is an isometric, cross-sectional, front view of the access port for joining of the trimmed conductor embodiment depicted in FIG. 7C.

FIG. 8A depicts an isometric view of a completed assembly of an embodiment demonstrating the installation of clamping fasteners.

FIG. 8B depicts an isometric, cross-sectional view of the completed assembly of the embodiment depicted in FIG. 8A.

### DETAILED DESCRIPTION

One or more illustrative embodiments incorporating the invention disclosed herein are presented below. The inventor has created a revolutionary and novel neurological stimulation and signal recording apparatus.

In the following description, certain details are set forth such as specific quantities, sizes, etc. to provide a thorough understanding of the present embodiments disclosed herein. However, it will be evident to those of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Referring to the drawings in general, it will be understood that the illustrations are for the purpose of describing embodiments of the disclosure and are not intended to be limiting thereto. Drawings are not necessarily to scale and arrangements of specific units in the drawings can vary.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless, or essentially meaningless, the definition should be taken from Webster's Dictionary 2023. Definitions and/or interpretations should not be incorporated from other patent applications, patents, or publications, related or not, unless specifically stated in this specification, or if the incorporation is necessary for maintaining validity.

While preferred embodiments have been shown, and described, modifications thereof can be made by one skilled in the art without departing from the scope or teaching herein. The embodiments described herein are exemplary only and are not limiting. Many variations and modifications of the system and apparatus are possible and will become apparent to those skilled in the art once the above disclosure is fully appreciated. For example, the relative dimensions of various parts, the materials from which the various parts are made, and other parameters can be varied.

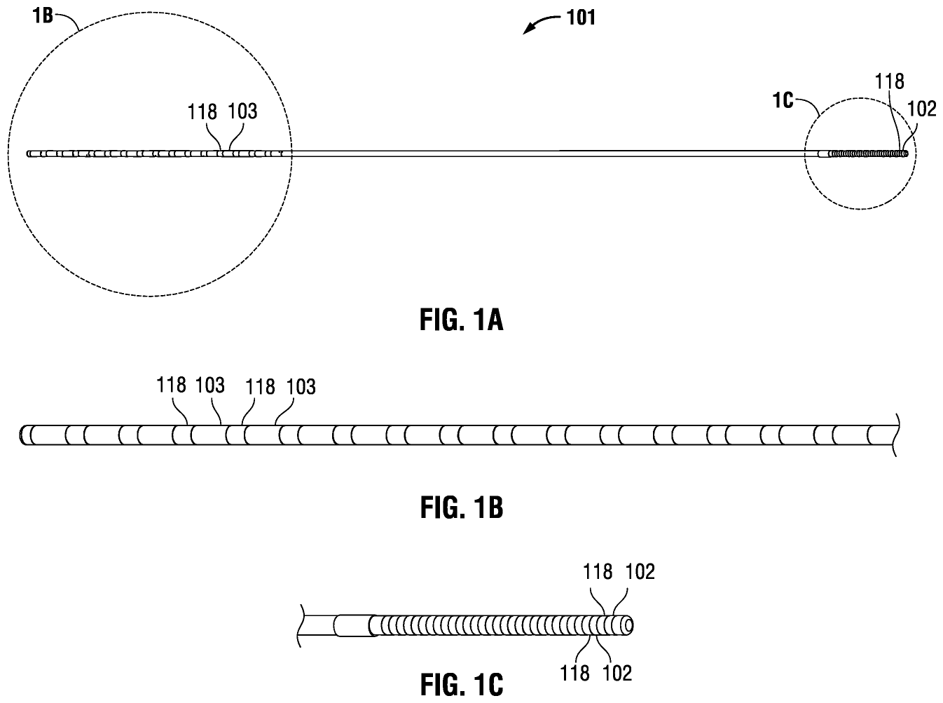

With reference to FIG. 1, the apparatus depicted is an embodiment of an isodiametric neurostimulation lead 101 comprising one or more electrodes 102 and one or more terminal bands 103. It should be appreciated that the electrodes 102 and terminal bands 103 are comprised of electrically conductive material to enable electrical communication between a respective electrode and terminal band via a conductor. The one or more electrodes 102 may be separated or spaced by the introduction of a polymer material 118 to a substructure of the isodiametric neurostimulation lead 101. Such substructure may include a substructure polymer. Such substructure polymer may allow for an over mold of polymer wherein the one or more terminals bands 103 and the one or more electrodes 102 are left uncoated by the polymer material in order to allow for conductivity of the one or more electrodes 102.

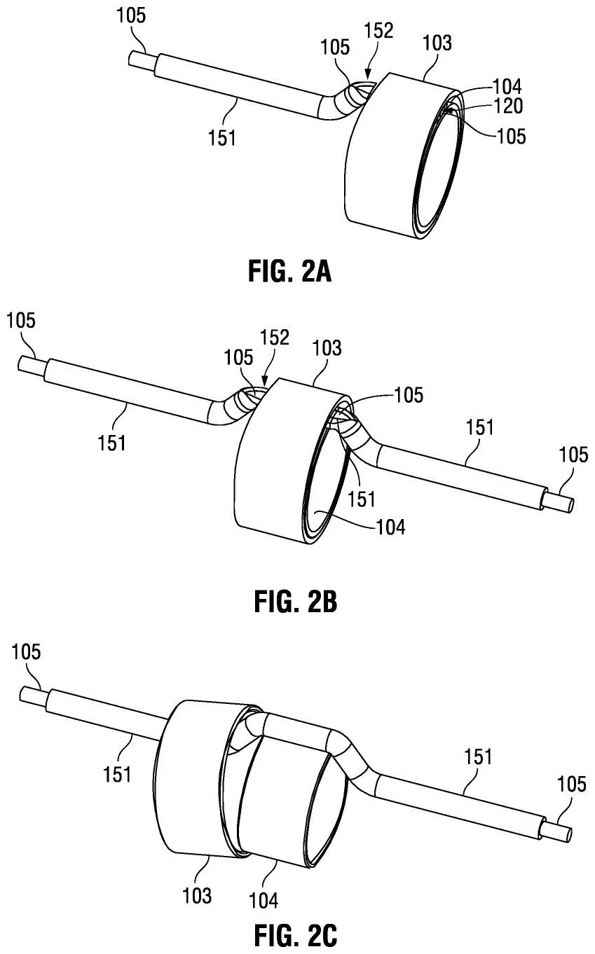

With reference to FIGS. 2A, 2B, and 2C, the one or more terminal bands 103 comprising an annulus having an exterior surface and interior surface and the one or more electrodes 102 may be electrically and mechanically joined by introducing inner band 104 comprising an annulus into the terminal band 103 annulus each having an exterior surface and interior surface. When inner band 104 is inserted into the terminal band 103, the exterior surface of the inner band 104 presses a conductor 105, typically insulated by an insulative material 151, against the interior surface of the terminal band 103. This process of sandwiching or joining the inner band 104 and terminal band 103 may in and of itself cause a portion of the insulative material 151 to strip away from conductor 105, thereby allowing electrical connectivity 152 of the conductor 105 to terminal band 103. Alternatively, a portion of the insulative material 151 may be removed before the sandwiching or joining of the inner band 104 and terminal band 103 to effect the electrical connectivity 152 between the conductor 105 and the terminal band 103. Some embodiments may use friction forces or narrow tolerances to secure the electrical connectivity 152 between the conductor 105 and the terminal band 103. Alternatively, some embodiments may include the use of an electrically conductive fastener, adhesive, weld, laser weld, resistance spot weld, and/or solder 120 to secure the electrical connectivity 152 between the conductor 105 and terminal band 103. Alternatively, some embodiments may include the use of a non-electrically conductive fastener, adhesive, weld, laser weld, resistance spot weld, and/or solder 120 to secure the electrical connectivity 152 between the conductor 105 and terminal band 103. In some embodiments, such as those depicted by FIG. 2C, the conductor 105 may be fully insulated with an insulation material 151 at the length of conductor 105 where it is to be sandwiched or joined between terminal band 103 and inner band 104. The process of sandwiching or joining terminal band 103 and inner band 104 with conductor 105 may strip away insulative material 151 as shown in FIG. 2B resulting in an electrical connectivity 152 between the terminal band 103 and conductor 105. In some embodiments, such as that depicted in FIG. 2A, the conductor 105 may be trimmed or cut on one end and then secured 120 between inner band 104 and terminal band 103, as described above. It should be appreciated that FIG. 2C is the first step of the process of assembling a terminal band 103, inner band 104, and conductor 105, FIG. 2B is the intermediate step, and FIG. 2A depicts a completed assembly of the terminal band, inner band, and conductor before the introduction of a polymer material substrate, as discussed herein. It should be appreciated that the same method may be applied to create the plurality electrode bands 102, inner bands 104, and conductors 105 of the present isodiametric neurostimulation lead.

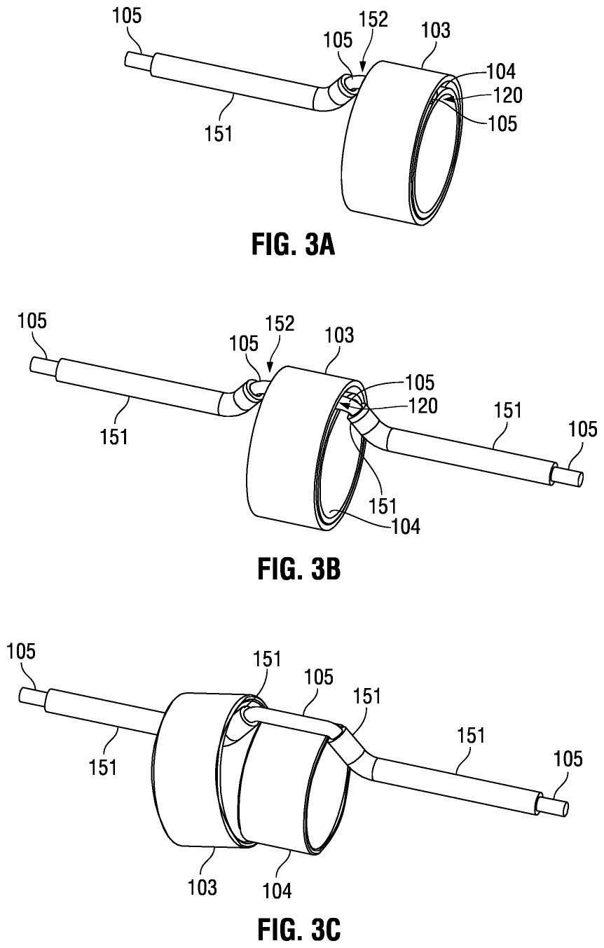

FIGS. 3A, 3B, and 3C depict an alternative embodiment of the isodiametric neurostimulation lead wherein the one or more terminal bands 103 comprising an annulus having an exterior surface and interior surface and the one or more electrodes 102 may be electrically and mechanically joined by introducing inner band 104 comprising an annulus also having an exterior surface and interior surface. When inner band 104 is inserted into the terminal band 103, the exterior surface of the inner band 104 presses a conductor 105, typically insulated by an insulative material 151, against the interior surface of the terminal band 103. This process of sandwiching or joining the inner band 104 and terminal band 103 may in and of itself cause a portion of the insulative material 151 to strip away from conductor 105, thereby allowing electrical connectivity 152 of the conductor 105 to terminal band 103. FIGS. 3A, 3B, and 3C depict an alternative embodiment wherein at least a portion of the insulative material 151 is removed before the sandwiching or joining of the inner band 104 and terminal band 103 to effect the electrical connectivity 152 between the conductor 105 and the terminal band 103 and inner band 104. Some embodiments may use friction forces or narrow tolerances to secure the electrical connectivity 152 between the conductor 105 and the terminal band 103. Alternatively, some embodiments may include the use of an electrically conductive fastener, adhesive, weld, laser weld, resistance spot weld, and/or solder 120 to secure the electrical connectivity 152 between the conductor 105 and terminal band 103. Alternatively, some embodiments may include the use of a non-electrically conductive fastener, adhesive, weld, laser weld, resistance spot weld, and/or solder 120 to secure the electrical connectivity 152 between the conductor 105 and terminal band 103. In some embodiments, such as those depicted by FIG. 3C, the conductor 105 may be fully striped of the insulation material 151 at the length of conductor 105 where it is to be sandwiched or joined between terminal band 103 and inner band 104. As shown in FIG. 3B, the pre-stripping of insulative material 151 allows for an electrical connectivity 152 between the terminal band 103, inner band 104, and conductor 105. In some embodiments, such as that depicted in FIG. 3A, the conductor 105 may be trimmed or cut on one end and then secured 120 between inner band 104 and terminal band 103, as described above. It should be appreciated that FIG. 3C is the first step of the process of assembling a terminal band 103, inner band 104, and conductor 105, FIG. 3B is the intermediate step, and FIG. 3A depicts a completed assembly of the terminal band, inner band, and conductor before the introduction of a polymer material substrate, as discussed herein.

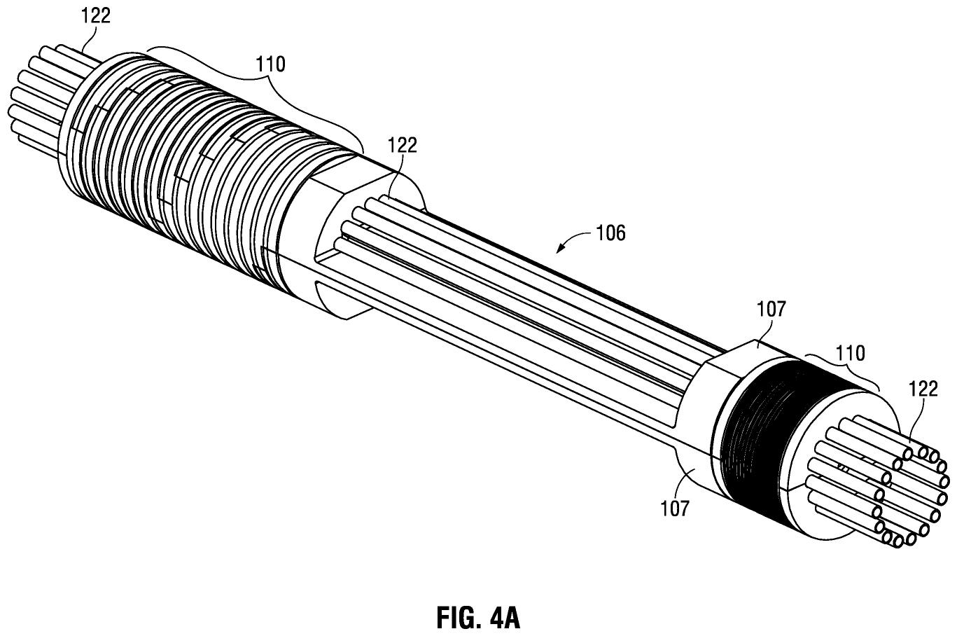

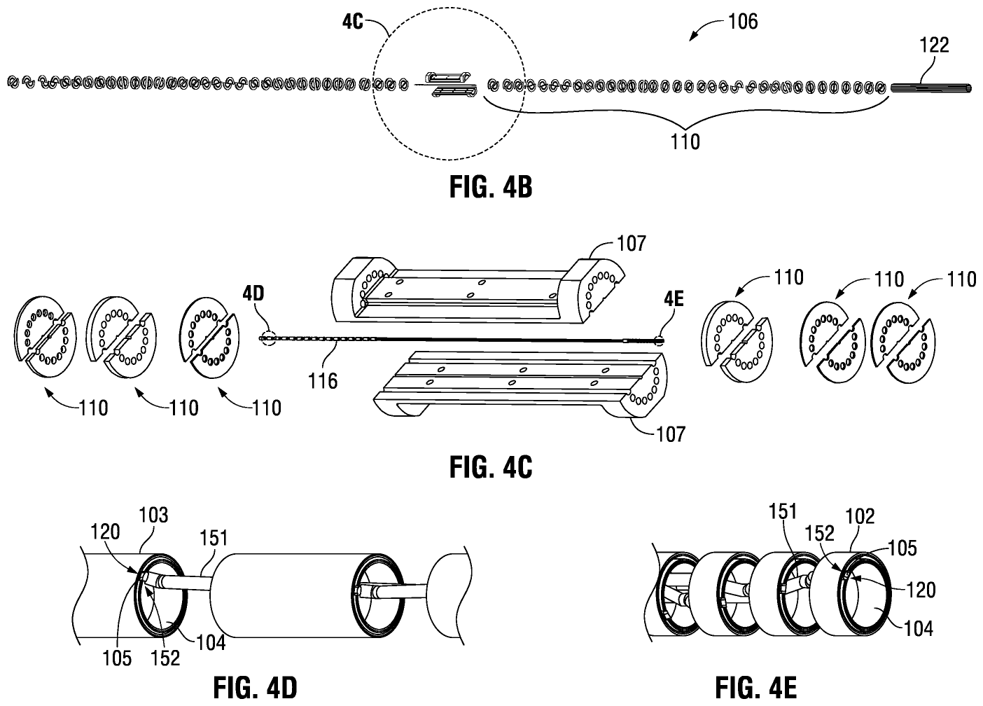

FIG. 4A depicts an apparatus mold assembly fixture 106 and represents the method and design for fabrication of the aforementioned isodiametric neurostimulation lead. A plurality of plates 110 are guided and secured in place through the use of guide pins 122. It should be appreciated that the guide pins 122 may be solid cylinders or hollow cylinders. It should be appreciated that the guide pins are not necessarily required to be round to achieve the same function. It should be appreciated that in some embodiments the individual plates comprising the plurality of plates 110 may comprise semi-circular portions having cavities formed through the facial surface of the plate. The plurality plates 110 define the spacing between the terminal bands 103 and electrodes 102. The structure and positioning of the plates 110 creates supports and standoffs. In some embodiments, the plurality of plates 110 are rotated one guide pin 122 position, clockwise or counterclockwise, after completion of each conductor assembly. FIG. 4B is an exploded view of the apparatus mold assembly fixture 106. It should be appreciated that FIG. 4B depicts the rotational offset of the plurality of plates 110 from each other as described above. FIG. 4C depicts an exploded view of mold assembly fixture 106, mold assembly body 107, a plurality of plates 110, and a completed conductor lead 116. FIG. 4D depicts terminal band 103 and inner band 104 sandwiching conductor 105, which is jacketed by insulation material 151 and having a portion of the insulation material removed to allow electrical connectivity between the conductor 105 and the terminal band 103. Such conductor 105 and terminal band 103 may be secured to each other at point 120, in a number of different manners as described herein. It should be appreciated that FIG. 4D depicts the rotational offset between the terminal bands and their respective joinders 120 to one or more conductors 105. FIG. 4E depicts electrode 102 and inner band 104 sandwiching conductor 105, which is jacketed by insulation material 151 and having a portion of the insulation material removed to allow electrical connectivity between the conductor 105 and the electrode 102. Such conductor 105 and electrode 102 may be secured to each other at point 120, in a number of different manners as described herein. It should be appreciated that FIG. 4E depicts the rotational offset between the electrodes and their respective joinders 120 to one or more conductors 105.

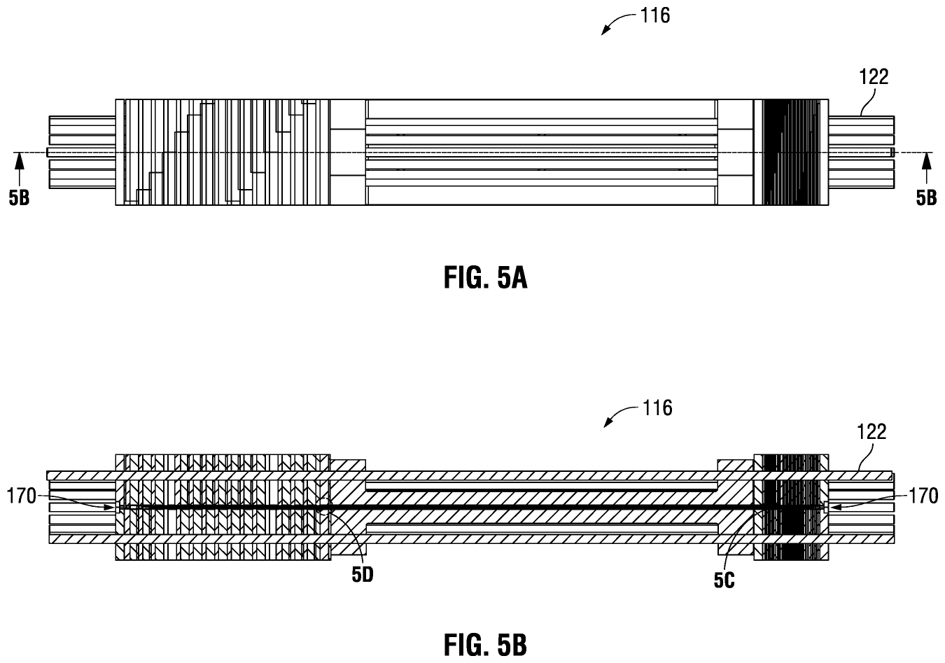

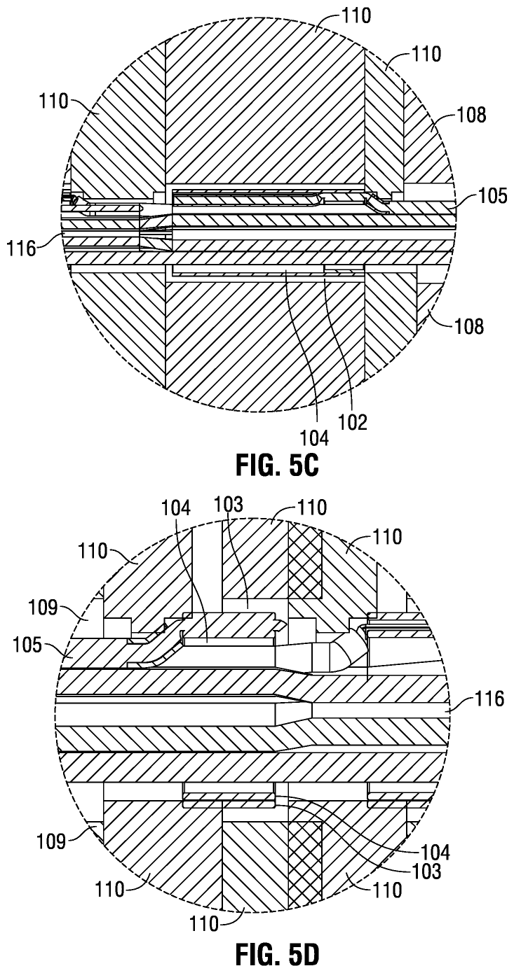

FIGS. 5A, 5B, 5C, and 5D depict an embodiment showing cavity 170 traversing a mold assembly fixture 106 wherein said conductor lead is positioned in a substantially centered position within the mold assembly fixture 106 and cavity 170. The mold assembly fixture 106 includes guide pins 122 to provide rigidity and stability for the body and plate assemblies. As is discussed herein, cavity 170 may be used to back fill or inject a polymer material into the cavity created by the plate assemblies of the mold assembly fixture. FIG. 5B is a cross-sectional depiction of the mold assembly fixture showing cavity 170 traversing the length thereof. FIG. 5C depicts a cross-sectional view of the electrode end of the completed conductor lead 116 within the mold assembly fixture. As shown, electrode band 102 and inner band 104 are sandwiching conductor 105, to allow electrical connectivity between conductor 105 and electrode band 102. Also shown are a plurality of plates 108 and 110 sandwiched or compressed together to create cavity 170. FIG. 5D depicts a cross-sectional view of the terminal end of the described conductor lead within the mold assembly fixture having plate assemblies. As shown, terminal band 103 and inner band 104 are sandwiching conductor 105, to allow electrical connectivity between the conductor 105 and the terminal band 103. Also shown are a plurality of plates 109 and 110 sandwiched or compressed together to create cavity 170. Also shown is completed conductor lead 116.

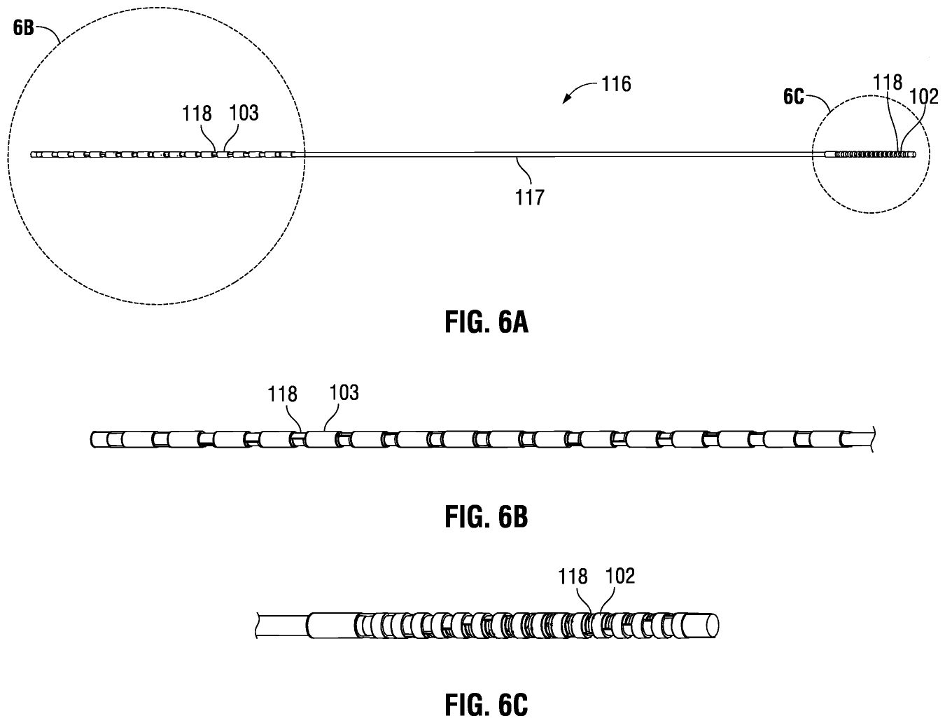

FIG. 6A shows a completed conductor lead 116 comprising a polymer substructure 117 formed via mold assembly fixture 106. The polymer substructure 117 is placed into an isodiametric mold and may be backfilled or injected with polymer material 118. The polymer substructure 117 may also be placed inside a thermal plastic tubing comprising a similar or equivalent polymer, then placed into an isodiametric shrink tube and thermally reflowed into one polymer structure, this over molding or reflow is then centerless ground to the desired diameter, after shrink tubing has been removed. FIG. 6B shows a detailed view of the terminal band 103 portion of the conductor lead 116 showing the interspersed plurality of terminal bands 103 with polymer material 118 separating each. FIG. 6C shows a detailed view of the electrode 102 portion of the conductor lead 116 showing the interspersed plurality of electrodes 102 with polymer material 118 separating each. The polymer separation is important to the various embodiments so as to electrically isolate each electrode and terminal band from the other neighboring electrodes or terminal bands. It should also be appreciated that in some embodiments each individual electrode may correspond to a specific individual terminal band through an electrical communication channel, such as conductor 105.

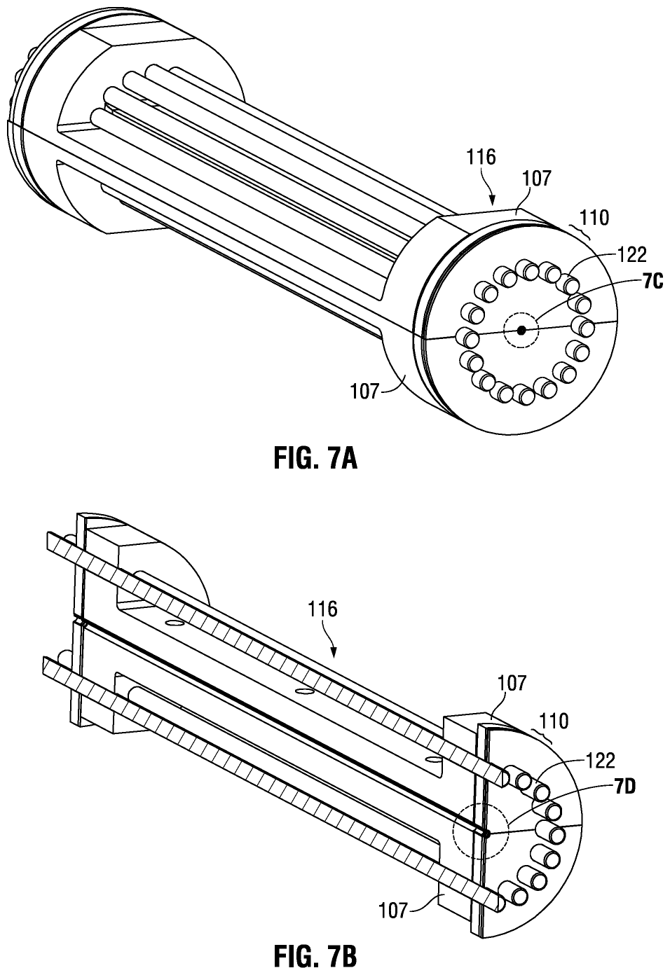

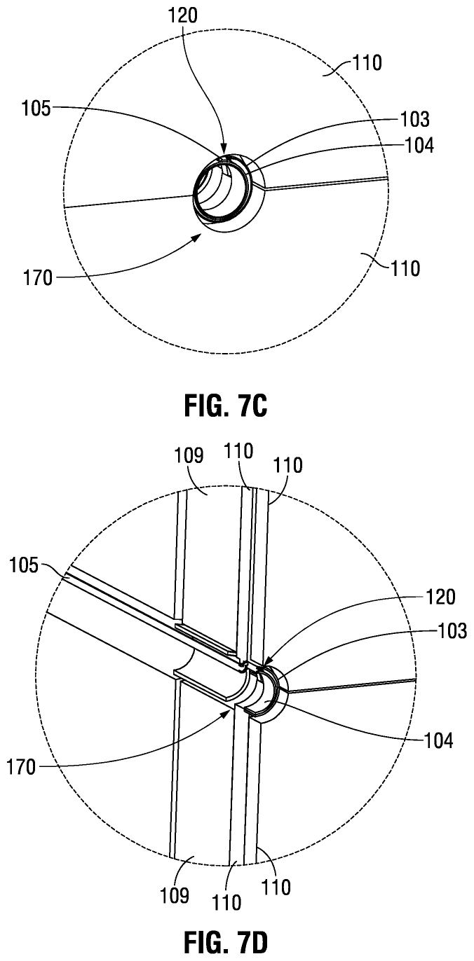

FIGS. 7A and 7C depict the mold assembly fixture 106, whereas FIGS. 7B and 7D depict a cut-away view of the same mold assembly fixture 106. Shown are guide pins 122 and mold assembly body 107, plates 110, end plate 109. In detail view FIG. 7C shown is a face of 2 semi-circular plates 110 forming a cavity 170 therebetween. Formed within said cavity 170 are terminal band 103, inner band 104, conductor 105, and joining point 120, whereby conductor 105 is in electrical communication with terminal band 103. FIG. 7D is a cutaway sideview of FIG. 7C allowing more detail to be shown. FIG. 7D depicts the additional plates 110 in a stacked formation in addition to end plate 109. Again, as in FIG. 7C and as described throughout, the plates 110 and end plate 109 form a cavity 170 therebetween. Formed within said cavity 170 are terminal band 103, inner band 104, conductor 105, and joining point 120, whereby conductor 105 is in electrical communication with terminal band 103. It should be appreciated that the disclosure is equally applicable to the formation of the electrode portion of the conductor lead.

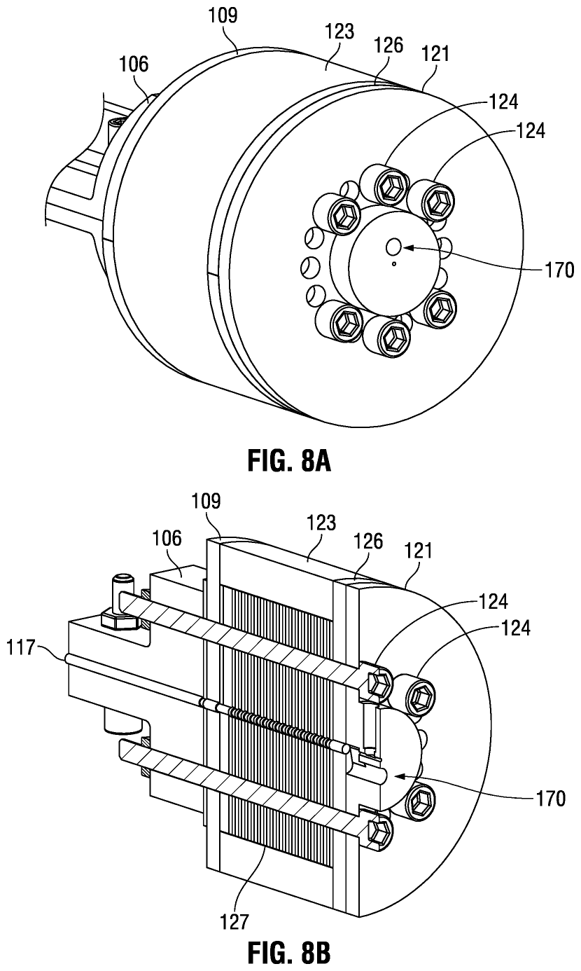

FIG. 8A depicts an isometric view of a final assembly embodiment and cross-sectional view of the same is depicted via FIG. 8B. The final assembly includes clamping fasteners 124 to securely maintain the assembly and components. It should be appreciated that a number of different fasteners known in the art may be used to securely maintain the assembly. The assembly includes a spacer component 123, which restricts the maximum height of the plate assembly 127 after the fasteners 124 have been installed and secured into the assembly. The plate assembly 127 comprises a plurality of plates 110. Spacer component 123 in some embodiments is optional provided that plate assembly 127 equals the length of spacer component 123. The spacer component 123 may in some embodiments includes stepped levels along an inner surface to allow for micro adjustments of the individual plates 110 comprising the plate assembly 127. Spacer component 123 in some embodiments can be milled, printed, or otherwise custom fitted to create the desired offset and spacing for the individual plates comprising the plurality of plates making up plate assembly 127. End plates 109, 121, and 126 may be used to sandwich the internal plate assembly 127 and spacer component 123. It should be appreciated that some plates 110 within the plate assembly 127 may be curved or warped with respect to a flat reference surface (not shown), any curvature or warping can be reduced or eliminated upon installing and securing the fasteners 124. The tolerances of plate assembly 127 and plurality of plates 110 may be assembled in a clockwise or counterclockwise rotation around the center axis as well as by controlling the orientation of any curved surfaces of the plates 110.

While preferred embodiments have been shown and described, modifications thereof can be made by one skilled in the art without departing from the scope or teaching herein. The embodiments described herein are exemplary only and are not limiting. Many variations and modifications of the system and apparatus are possible and will become apparent to those skilled in the art once the above disclosure is fully appreciated. For example, the relative dimensions of various parts, the materials from which the various parts are made, and other parameters can be varied. Further, it should be appreciated that the disclosure and teachings of the several embodiments described herein may be used interchangeably to achieve different embodiments not explicitly depicted. Accordingly, it is intended that the following claims be interpreted to embrace all such variations and modifications.

What is claimed is the following:

1. A neurostimulation lead comprising:
a lead body comprising a terminal end and an electrode end;
said terminal end is comprised of a terminal band, an inner band, an electrical conductor, wherein said electrical conductor has a proximal end and a distal end, wherein said terminal band has an inner surface and outer surface and said inner band has an outer surface, wherein said proximal end of said electrical conductor is in electrical communication with said terminal band when said electrical conductor is disposed between said terminal band and said inner band;
said electrode end is comprised of an electrode band, an inner band, and the distal end of said electrical conductor, wherein said electrode band has an inner surface and outer surface and said inner band has an outer surface, wherein said distal end of said electrical conductor is in electrical communication with said terminal band when said electrical conductor is disposed between said electrode band and said inner band.

2. The neurostimulation lead of claim 1 wherein said electrical conductor is coated with an electrically insulative material.

3. The neurostimulation lead of claim 1 further comprising:
a polymer substrate.

4. The neurostimulation lead of claim 2 further comprising:
a polymer substrate.

5. The neurostimulation lead of claim 2 wherein a portion of the electrically insulative material coating of said electrical conductor is removed from the electrical conductor by the mating of said inner surface of said terminal band with said outer surface of said inner band thereby facilitating an electrical connection between said proximal end of said electrical conductor and said terminal band.

6. The neurostimulation lead of claim 2 wherein a portion of the electrically insulative material coating of said distal end of said electrical conductor is removed from the electrical conductor by the mating of said inner surface of said electrode band with said outer surface of said inner band thereby facilitating an electrical connection between said distal end of said electrical conductor and said electrode band.

7. The neurostimulation lead of claim 5 wherein a portion of the electrically insulative material coating of said distal end of said electrical conductor is removed from the electrical conductor by the mating of said inner surface of said electrode band with said outer surface of said inner band thereby facilitating an electrical connection between said distal end of said electrical conductor and said electrode band.

8. The neurostimulation lead of claim 2 wherein a portion of the electrically insulative material coating of said electrical conductor is removed before assembly of said electrical conductor, said terminal band, and said inner band thereby facilitating an electrical connection between said proximal end of said electrical conductor and said terminal band.

9. The neurostimulation lead of claim 2 wherein a portion of the electrically insulative material coating of said electrical conductor is removed before assembly of said electrical conductor, said electrode band, and said inner band thereby facilitating an electrical connection between said distal end of said electrical conductor and said electrode band.

10. The neurostimulation lead of claim 8 wherein a portion of the electrically insulative material coating of said electrical conductor is removed before assembly of said electrical conductor, said electrode band, and said inner band thereby facilitating an electrical connection between said distal end of said electrical conductor and said electrode band.

11. The neurostimulation lead of claim 7 further comprising a polymer substrate.

12. The neurostimulation lead of claim 10 further comprising a polymer substrate.

13. The neurostimulation lead of claim 1 wherein said electrical conductor is fastened to said terminal band or said electrode band by one of the following: weld, laser weld, resistance spot weld, or solder.

14. The neurostimulation lead of claim 10 wherein said electrical conductor is fastened to said terminal band or said electrode band by one of the following: weld, laser weld, resistance spot weld, or solder.

15. A neurostimulation lead comprising:
a lead body comprising a proximal end and a distal end;
a plurality of terminal bands disposed at the proximal end of said lead body;
a plurality of electrode bands disposed at the distal end of said lead body;
a plurality of electrical conductors electrically connecting each of said terminal bands to a respective one of said electrode bands;
a polymer substrate isolating the plurality of terminal bands from each other terminal band and further isolating the plurality of electrode bands from each other electrode band and further isolating the plurality of electrical conductors from each other electrical conductors to form a plurality of electrically communicative terminal bands with respective electrode bands.

16. The neurostimulation lead of claim 15 wherein said electrical conductor is fastened to said terminal bands or said electrode bands by one of the following: weld, laser weld, resistance spot weld, or solder.

17. A method of manufacturing a neurostimulation lead comprising the steps of:
electrically connecting a terminal end of a lead body with an electrode end of said lead body, wherein said terminal end is comprised of a terminal band, an inner band, an electrical conductor, wherein said electrical conductor has a proximal end and a distal end, wherein said terminal band has an inner surface and outer surface and said inner band has an outer surface, wherein said proximal end of said electrical conductor is in electrical communication with said terminal band when said electrical conductor is disposed between said terminal band and said inner band; wherein, said electrode end is comprised of an electrode band, an inner band, and the distal end of said electrical conductor, wherein said electrode band has an inner surface and outer surface and said inner band has an outer surface, wherein said distal end of said electrical conductor is in electrical communication with said terminal band when said electrical conductor is disposed between said electrode band and said inner band;
placing said lead body into a mold assembly fixture having a cavity;
placing a plurality of plates on said terminal end of said mold assembly fixture;
placing a plurality of plates on said electrode end of said mold assembly fixture;
insertion of guide pins through mold assembly fixture and said plurality of plates on said terminal end and said electrode end of said mold assembly fixture;
placement of a spacer component around said plurality of plates at said terminal end or said electrode end to provide offsets or alignment for said plurality of plates;
injecting a polymer material into said cavity of said mold assembly fixture to form a polymer substructure around the electrically connected terminal end of said lead body and said electrode end of said lead body.

18. The method of manufacturing said neurostimulation lead of claim 17 wherein said electrical conductor is fastened to said terminal bands or said electrode bands by one of the following: weld, laser weld, resistance spot weld, or solder.

19. The method of manufacturing said neurostimulation lead of claim 17 further comprising the step of injecting another layer of polymer material to provide an overmold layer of polymer material.

20. The neurostimulation lead of claim 16 further comprising a second layer of polymer material over said polymer substrate.
